(19) United States
(12) Patent Application Publication
(10) Pub. No.: US 2025/0242135 A1
(43) Pub. Date: Jul. 31, 2025

(54) ALIGNMENT METHODS AND SYSTEMS FOR AORTIC CROSSING

(71) Applicant: Medtronic Vascular, Inc., Santa Rosa, CA (US)

(72) Inventors: Paraic G. FRISBY, Galway (IE); Ian K. LALOR, Galway (IE); Kate MOLAN, Galway (IE); Julie-Ann McBARRON, Galway (IE); Lara M. FRAMPTON, Galway (IE); Niall F. DUFFY, Galway (IE); Timothy D. FARRELL, Galway (IE); John M. CURLEY, Galway (IE)

(21) Appl. No.: 19/034,965

(22) Filed: Jan. 23, 2025

Related U.S. Application Data
(60) Provisional application No. 63/625,343, filed on Jan. 26, 2024.

(51) Int. Cl. A61M 25/09; A61B 18/00

(52) U.S. Cl. CPC A61M 25/0905; A61B 18/00; A61B 2018/00369

(57) ABSTRACT

An aortic method for aortic valve crossing is disclosed. The method includes advancing a positioning catheter through an aorta to a delivery position in which a distal end of the positioning catheter is spaced apart from an aortic valve crossing. The positioning catheter defines a positioning lumen containing positioning arms. The method further includes landing the positioning arms on cusps of aortic leaflets of the aortic valve crossing to align the positioning catheter with an opening defined by the aortic leaflets. The method also includes extending a longitudinally extending guidewire through the opening.

### CROSS-REFERENCE TO RELATED APPLICATIONS

This application claims the benefit of U.S. Provisional Patent Application Ser. No. 63/625,343, filed Jan. 26, 2024, the entire content of which is incorporated herein by reference.

### TECHNICAL FIELD

The present application is generally related to alignment methods and systems for aortic crossing.

### BACKGROUND

Prosthetic heart valve designs aim to mimic the function of natural heart valve designs. One type of prosthetic heart valve design is referred to as a self-expanding valve (e.g., a supra-annular design). The self-expanding valve design includes an expandable stent frame formed of a self-expanding material (e.g., a nickel titanium alloy or Nitinol). Prosthetic leaflets may be mounted onto the expandable stent frame to replicate natural aortic leaflets.

Prosthetic heart valve delivery systems are delivered using procedures which may utilize several guidewires and catheters to provide a guided path for delivery of the prosthetic heart valve to an implementation site. This guided path extends through the aortic leaflets and typically includes guidewires and catheters extending through the opening defined by the aortic leaflets. Stenosed aortic leaflets do not open as much as healthy aortic leaflets. Therefore, applying the prosthetic heart valve delivery procedure to stenosed aortic leaflets may be challenging due to the difficulty of crossing the stenosed aortic leaflets with guidewires and catheters.

### SUMMARY

In a first embodiment, an alignment method for aortic valve crossing is disclosed. The method includes advancing a positioning catheter through an aorta to a delivery position in which a distal end of the positioning catheter is spaced apart from an aortic valve crossing. The positioning catheter defines a positioning lumen containing positioning arms. The method further includes landing the positioning arms on cusps of aortic leaflets of the aortic valve crossing to align the positioning catheter with an opening defined by the aortic leaflets. The method also includes extending a longitudinally extending guidewire through the opening.

The landing step may include extending the positioning arms through the distal end of the positioning catheter such that the positioning arms extend outward the positioning catheter to contact the cusps of the aortic leaflets. The extending step may include curling the distal ends of the positioning arms to contact the cusps of the aortic leaflets atraumatically. The landing step may include advancing the positioning catheter to invert the positioning arms about joints positioned medially the positioning arms. The advancement of the positioning catheter extends the longitudinally extending guide through the opening. The landing step may include manipulating each of the positioning arms independently to align the positioning catheter with the opening defined by the aortic leaflets. The manipulating step may include tensioning the positioning catheter.

In a second embodiment, an alignment method for aortic valve crossing is disclosed. The method includes advancing a positioning sheath through an aorta to a delivery position in which a distal end of the positioning sheath is spaced apart from an aortic valve crossing. The positioning sheath defines a positioning lumen containing positioning arms. The method includes retracting the positioning sheath to expose the positioning arms. The method further includes landing the positioning arms on cusps of aortic leaflets of the aortic valve crossing to align the positioning sheath with an opening defined by the aortic leaflets. The method also includes extending a longitudinally extending guidewire through the opening.

The positioning arms include proximal ends and distal ends. The distal ends are attached to a positioning catheter extending within the positioning lumen. The landing step may include advancing the positioning catheter such that the positioning arms contact the cusps of the aortic leaflets. The longitudinally extending guidewire within a guide catheter. The positioning arms may include a first positioning arm, a second positioning arm, and a third positioning arm.

The aortic leaflets include a first aortic leaflet, a second aortic leaflet, and a third aortic leaflet. The landing step may include landing the first positioning arm on a first cusp of the first aortic leaflet, landing the second positioning arm on a second cusp of the second aortic leaflet, and landing the third positioning arm on a third cusp of the third aortic leaflet.

The landing step may include positioning the first, second, and third positioning arms in a tripod configuration. The tripod configuration forms a space for extension of the longitudinally extending guide.

In another embodiment, an alignment method for aortic valve crossing is disclosed. The method includes advancing a positioning sheath through an aorta to a delivery position in which a distal end of the positioning sheath is spaced apart from an aortic valve crossing. The positioning sheath defines a positioning lumen containing positioning arms. The method may further include landing the positioning arms on a portion of an ascending aorta to align the positioning catheter with an opening defined by aortic leaflets of the aortic valve crossing. The method further includes extending a longitudinally extending guidewire through the opening.

The positioning arms may be anchored to an outer surface of a positioning catheter. The landing step may include differentially moving the positioning sheath or the positioning catheter such that the positioning arms extend outward the positioning catheter to contact the portion of the ascending aorta. The differentially moving step includes advancing the positioning catheter relative to the positioning sheath. The differentially moving step may include retracting the positioning sheath relative to the positioning catheter. The positioning arms may include distal ends having spindles.

### BRIEF DESCRIPTION OF THE DRAWINGS

FIG. 1A depicts a cross-sectional view of a patient anatomy and a schematic, side view of a pre-curved guidewire extending through the patient anatomy.

FIG. 1B depicts a cross-sectional view of the patient anatomy and a schematic, side view of a delivery system tracking over the pre-curved guidewire in a pre-deployment state.

FIG. 1C depicts a cross-sectional view of the patient anatomy and a schematic, side view of a transcatheter aortic valve replacement (TAVR) device in a partially deployed position.

FIG. 1D depicts a cross-sectional view of the patient anatomy and a schematic, side view of the TAVR device in a fully deployed position.

FIG. 2A depicts a cross-sectional view of a patient anatomy and a schematic, side view of a guide catheter extending through the patient anatomy.

FIG. 2B depicts a top view of a stenosed valve in an open state.

FIG. 2C depicts a top view of a stenosed valve in a closed state.

FIG. 2D depicts a top view of a healthy valve in an open state.

FIG. 2E depicts a top view of a healthy valve in a closed state.

FIG. 3A depicts a cross-sectional view of a patient anatomy and a schematic, side view of a positioning catheter with positioning arms.

FIG. 3B depicts a cross-sectional view of the positioning catheter with positioning arms taken along line 3B-3B of FIG. 3A.

FIG. 3C depicts a cross-sectional view of a patient anatomy and a schematic, side view of a guide catheter extending through the positioning catheter.

FIG. 4A depicts a cross-sectional view of a patient anatomy and a schematic, side view of a positioning catheter and positioning arms in a first deployed position.

FIG. 4B depicts a cross-sectional view of the positioning catheter with positioning arms taken along line 4B-4B of FIG. 4A.

FIG. 4C depicts a cross-sectional view of a patient anatomy and a schematic, side view of a guide catheter extending through the positioning catheter in a second deployed position.

FIG. 5A depicts a top view of an aortic valve and positioning arms of a positioning sheath.

FIG. 5B depicts a side view of the positioning sheath showing the positioning arms in a constrained position.

FIG. 5C depicts a cross-sectional view of a patient's aortic valve crossing and a schematic, side view of the positioning sheath in a retracted position.

FIG. 5D depicts a cross-sectional view of a patient's aortic valve crossing and a schematic, side view of the positioning arms in a deployed position.

FIG. 6A depicts a cross-sectional view of a positioning sheath and a schematic, side view of a positioning catheter with positioning arms in a constrained position within the positioning sheath.

FIG. 6B depicts a cross-sectional view of the positioning sheath and a schematic, side view of the positioning catheter with the positioning arms in a deployed position outside of the positioning sheath.

FIG. 6C depicts a cross-sectional view of a patient anatomy and a schematic, side view of the positioning catheter with the positioning arms in the deployed position.

FIG. 7A depicts a cross-sectional view of an aortic wall and a schematic, side view of a positioning catheter with positioning spindles in a constrained position.

FIG. 7B depicts a cross-sectional view of an aortic wall and a schematic, side view of the positioning catheter with the positioning spindles in a deployed position.

FIG. 7C depicts a cross-sectional view of a patient anatomy and a schematic, side view of the positioning catheter with the positioning spindles in the deployed position.

### DETAILED DESCRIPTION

Embodiments of the present disclosure are described herein. It is to be understood, however, that the disclosed embodiments are merely examples and other embodiments can take various and alternative forms. The figures are not necessarily to scale; some features could be exaggerated or minimized to show details of particular components. Therefore, specific structural and functional details disclosed herein are not to be interpreted as limiting, but merely as a representative basis for teaching one skilled in the art to variously employ the embodiments. As those of ordinary skill in the art will understand, various features illustrated and described with reference to any one of the figures can be combined with features illustrated in one or more other figures to produce embodiments that are not explicitly illustrated or described. The combinations of features illustrated provide representative embodiments for typical applications. Various combinations and modifications of the features consistent with the teachings of this disclosure, however, could be desired for particular applications or implementations.

Directional terms used herein are made with reference to the views and orientations shown in the exemplary figures. A central axis is shown in the figures and described below. Terms such as “outer” and “inner” are relative to the central axis. For example, an “outer” surface means that the surfaces faces away from the central axis, or is outboard of another “inner” surface. Terms such as “radial,” “diameter,” “circumference,” etc. also are relative to the central axis. The terms “front,” “rear,” “upper” and “lower” designate directions in the drawings to which reference is made.

Unless otherwise indicated, for the delivery system the terms “distal” and “proximal” are used in the following description with respect to a position or direction relative to a treating clinician. “Distal” and “distally” are positions distant from or in a direction away from the clinician, and “proximal” and “proximally” are positions near or in a direction toward the clinician. For the stent-graft prosthesis, “proximal” is the portion nearer the heart by way of blood flow path while “distal” is the portion of the stent-graft further from the heart by way of blood flow path.

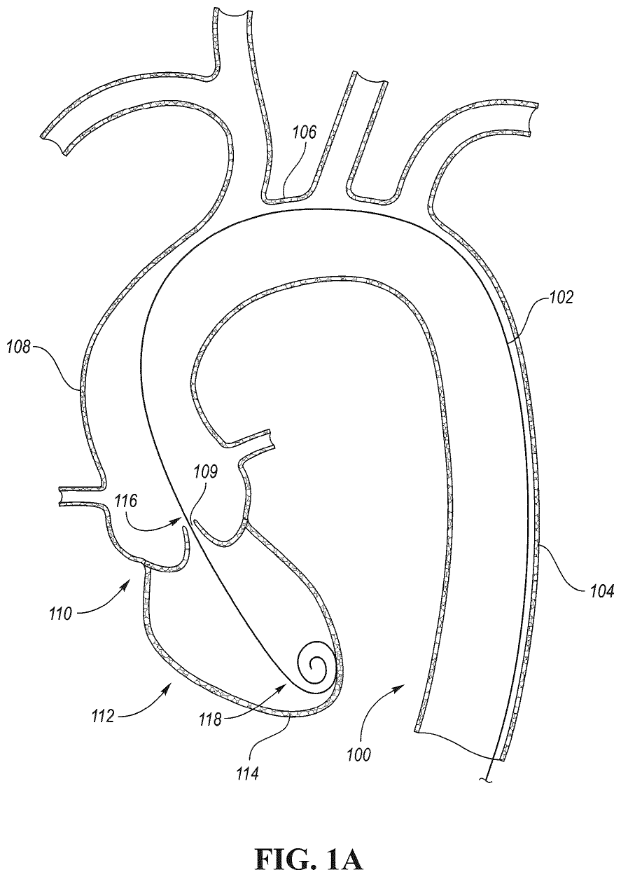

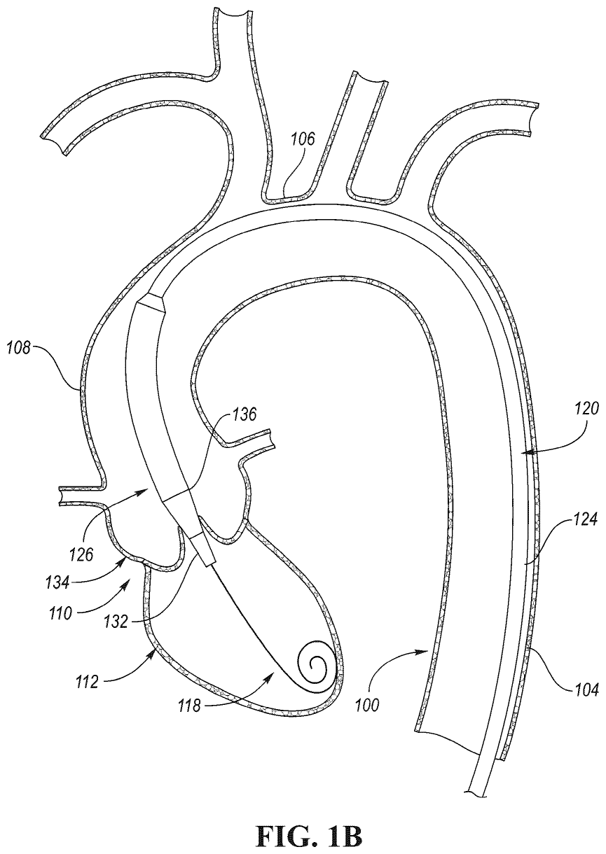

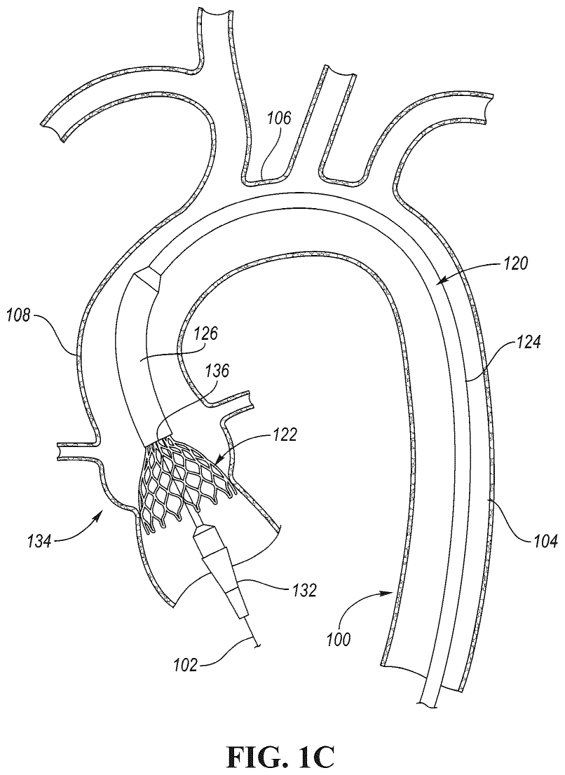

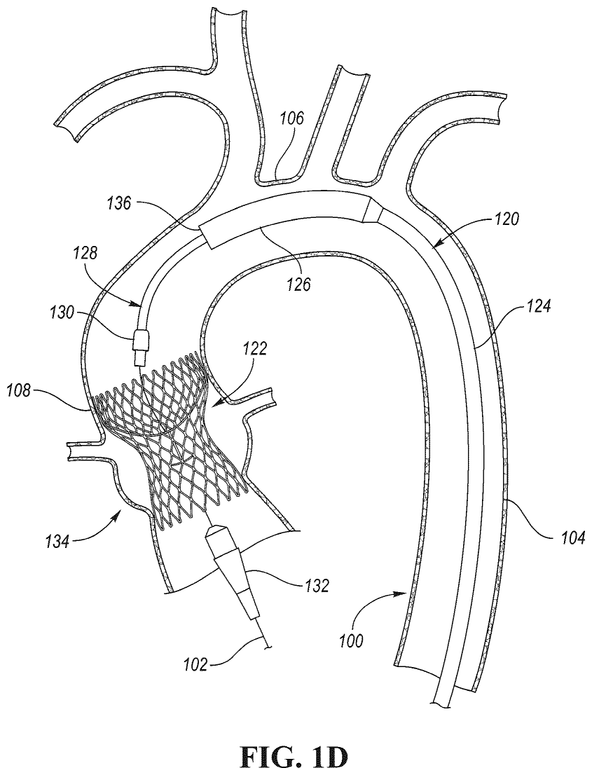

FIG. 1A depicts a cross-sectional view of patient anatomy 100 and a schematic, side view of pre-curved guidewire 102 extending through patient anatomy 100. Patient anatomy 100 includes descending aorta 104, aortic arch 106, ascending aorta 108, aortic leaflets 110, and heart 112 having heart wall 114. Descending aorta 104 is in communication with aortic arch 106. Aortic arch 106 is in communication with ascending aorta 108. Ascending aorta 108 is in communication with aortic leaflets 110 of an aortic valve.

Aortic leaflets 110 may be native aortic leaflets or replacement, prosthetic aortic leaflets. The replacement aortic leaflets may be a surgical aortic valve replacement (SAVR) device or a transcatheter aortic valve replacement (TAVR) device, such as a self-expanding TAVR device or a balloon inflatable TAVR device. Such devices generally include a frame (e.g., a stent frame) and a prosthetic valve. A TAVR device may be delivered to an implantation site using pre-curved guidewire 102 to track the TAVR device in a delivery state around patient anatomy 100 and through aortic leaflets 110.

Pre-curved guidewire 102 includes substantially straight portion 116 and pre-curved portion 118 extending from substantially straight portion 116. In one or more embodiments, pre-curve portion 118 forms a flexible tip region having greater flexibility than substantially straight portion 116, which may be considered a stiff region. Pre-curved portion may be a self-expanding structure formed of a metal alloy (e.g., stainless steel alloy, nickel titanium alloy, Nitinol, or other shape memory material).

As shown in FIG. 1A, pre-curved guidewire 102 extends through opening 109 defined by aortic leaflets 110 in a delivery position. The delivery position may be achieved using the following procedure. First, a straight tip guidewire is inserted into a patient's vasculature and advanced through opening 109 of aortic leaflets 110. Subsequently, a catheter (e.g., angiographic catheter) is placed over the straight tip guidewire and is advanced into heart 112 (e.g., a ventricle of heart 112). The straight tip guidewire is then removed from the catheter and the patient's vasculature, and replaced with a J-tip wire, which is inserted into the patient's vasculature and advanced through the catheter. The catheter may then be exchanged for a second catheter (e.g., pigtail catheter) by withdrawing the catheter and then inserting the second catheter into the patient's vasculature and advancing it over the catheter-tip wire. The J-tip wire is then exchanged for pre-curved guidewire 102 by advancement through the second catheter. While pre-curved guidewire 102 advances through the second catheter, pre-curved portion 118 is straightened by the second catheter in a constrained state. When pre-curved portion 118 emerges from the distal end of the second catheter, pre-curved portion 118 takes on its pre-curved shape as shown in FIG. 1A and may contact heart wall 114. The above process for establishing a curved guidewire in the left ventricle is merely an example, and other steps or devices may be used to establish the guidewire.

FIG. 1B depicts a cross-sectional view of patient anatomy 100 and a schematic, side view of delivery system 120 tracking over pre-curved guidewire 102 in a pre-deployment state. FIG. 1C depicts a cross-sectional view of patient anatomy 100 and a schematic, side view of delivery system 120 and TAVR device 122 in a partially deployed position. FIG. 1D depicts a cross-sectional view of patient anatomy 100 and a schematic, side view of delivery system 120 and TAVR device 122 in a fully deployed position.

Delivery system 120 includes delivery sheath assembly 124, capsule 126, inner shaft assembly 128, retention hub 130, and tip 132. Delivery system 120 is configured to retain TAVR device 122 in a delivery state in which TAVR device 122 is loaded in a constrained position within delivery system 120. In the delivery state, TAVR device 122 is coupled to inner shaft assembly 128 via retention hub 130 (e.g., TAVR device 122 may include eyelets captured by retention hub 130) and is compressively retained within capsule 126 of delivery sheath assembly 124. Loaded delivery system 120 may be configured to percutaneously deliver TAVR device 122 to implantation site 134 (e.g., a defective heart valve). As shown in FIGS. 1C and 1D, loaded delivery system 120 may be advanced toward implantation site over pre-curved guidewire 102 in a retrograde manner through the patient's femoral artery into descending aorta 104 over aortic arch 106 through ascending aorta 108 and across a defective heart valve (e.g., about mid-way through defective heart valve) at implantation site 134.

For self-expanding embodiments, delivery sheath assembly 124 is configured to withdraw capsule 126 proximally from TAVR device 122 via operation of a handle (not shown) on delivery system 120, thereby allowing TAVR device 122 to expand from the constrained, pre-deployment position to a partially deployed position shown in FIG. 1C and fully deployed position shown in FIG. 1D in which TAVR device 122 is fully released from capsule 126 and TAVR device 122 is implanted to the native valve. A release sheath assembly of delivery system 120 may be configured to fully release TAVR device 122 from capsule 126. The handle may be configured to maneuver capsule 126 to the partially deployed position shown in FIG. 1C in which a distal region of TAVR device 122 is permitted to self-expand at distal end 136 of retention hub 130 while a proximal region of TAVR device 122 remains coupled to retention hub 130.

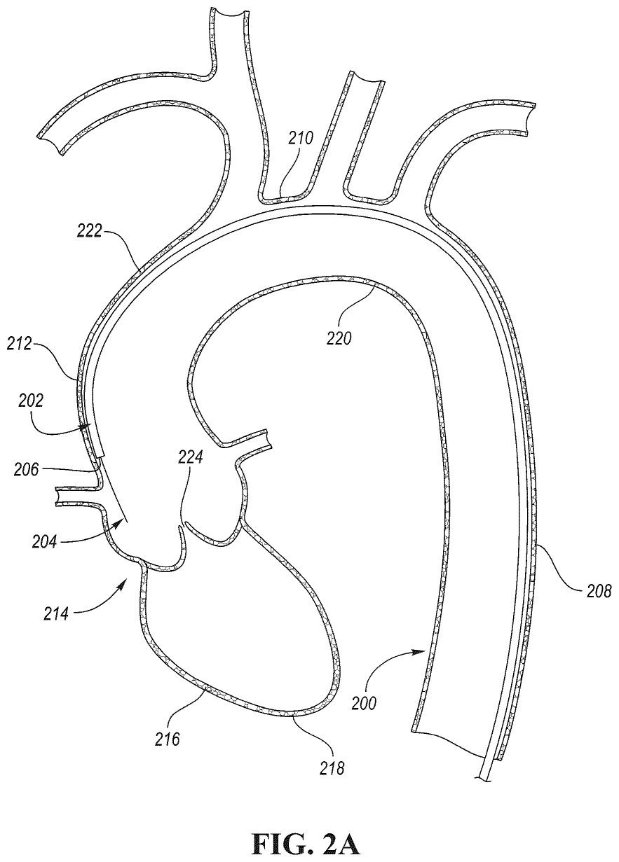

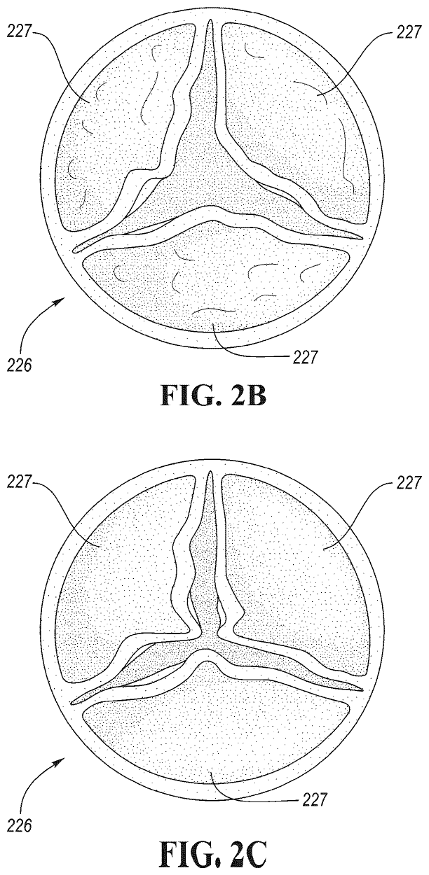

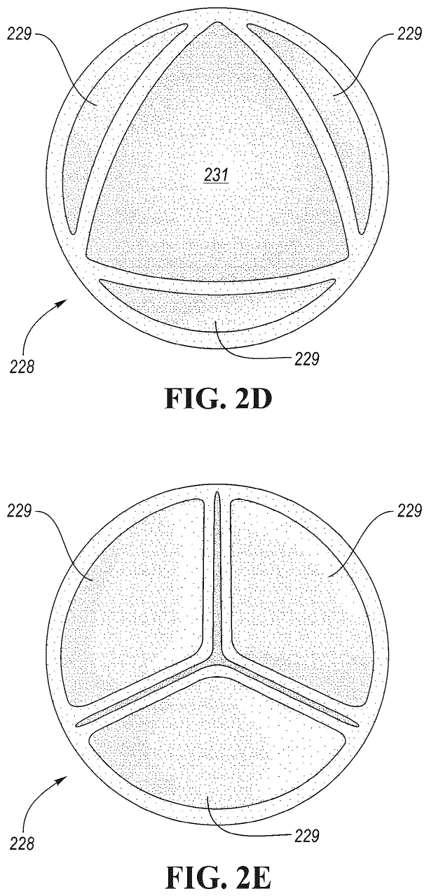

FIG. 2A depicts a cross-sectional view of patient anatomy 200 and a schematic, side view of guide catheter 202 extending through patient anatomy 200. As shown in FIG. 2A, straight tip guidewire 204 has extended through distal end 206 of guide catheter 202. Patient anatomy 200 includes descending aorta 208, aortic arch 210, ascending aorta 212, aortic leaflets 214, and heart 216 having heart wall 218. The aorta includes outer curve 222 and inner curve 220.

As shown in FIG. 2A, guide catheter 202 and straight tip guidewire 204 has aligned with outer curve 222 of the aorta. This alignment may occur due to the stiffness of the body of guide catheter 202 and straight tip guidewire 204 causing these components to bow outward toward outer curve 222. As shown in FIG. 2A, guide catheter 202 and straight tip guidewire 204 are not aligned with opening 224 defined by aortic leaflets 214. This misalignment may present a challenge to deployment of a TAVR device at an implantation site.

While misalignment may increase the amount of time taken to cross opening 224, one or more other factors may increase implantation procedure time. FIG. 2B depicts a top view of stenosed valve 226 in an open state. FIG. 2C depicts a top view of stenosed valve 226 in a closed state. Stenosed valve 226 includes stenosed aortic leaflets 227. FIG. 2D depicts a top view of healthy valve 228 in an open state. FIG. 2E depicts a top view of healthy valve 228 in a closed state. Healthy valve 228 includes healthy aortic leaflets 229. In healthy valve 228, blood flows out of heart 216 through opening 231 in the open state while heart 216 beats and closes substantially in the closed state. In the open state of healthy valve 228, opening 231 provides a large opening for crossing the annulus, although the force of blood flowing out of heart 216 works against catheter(s) and/or guidewire(s) crossing the annulus. However, crossing the annulus in the closed state with healthy valve 228 substantially closed presents difficulties. The difficulties are present in both the open and closed states of stenosed valve 226, where the stenosed aortic leaflets 227 do not fully open or close but are rather in a partially closed state. Another factor that may contribute to the difficulty in crossing the annulus is that the catheter(s) and/or guidewire(s) may get caught on the aortic leaflets. These difficulties may add to procedure time where reducing procedure time may enhance the benefit of the procedure.

Considering the foregoing, what is needed is an aortic leaflet crossing system and method to address one or more of the factors identified above that may contribute to increased TAVR device implantation procedure time. What is also needed is an aortic leaflet crossing system and method to align a guidewire prior to crossing the aortic leaflets. In one or more embodiments, aortic leaflet crossing systems are disclosed that may repeatedly and consistently cross aortic leaflets with a guidewire with proper alignment while reducing the instance of one or more of the challenging factors identified above.

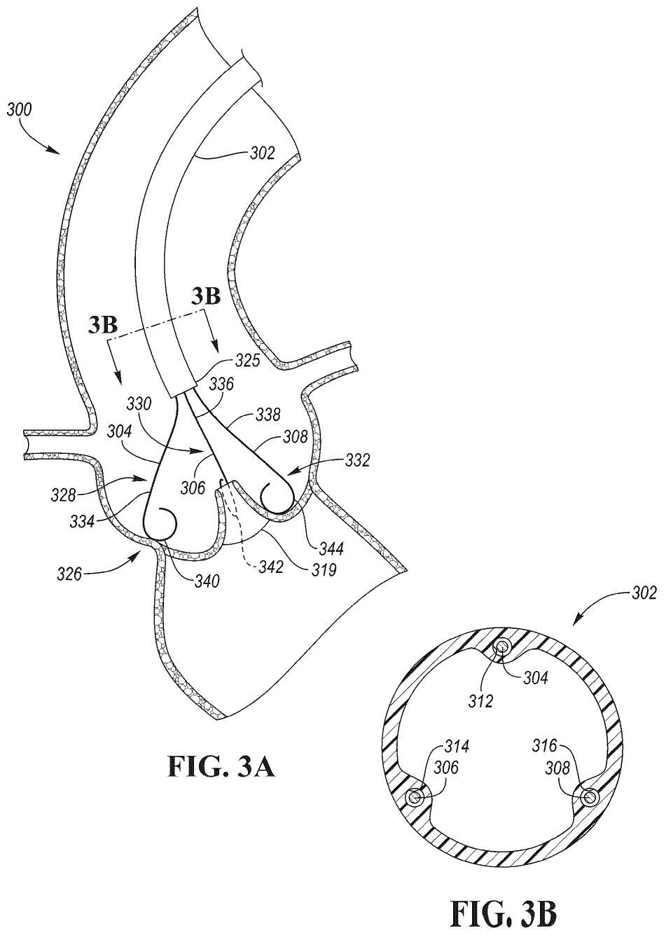

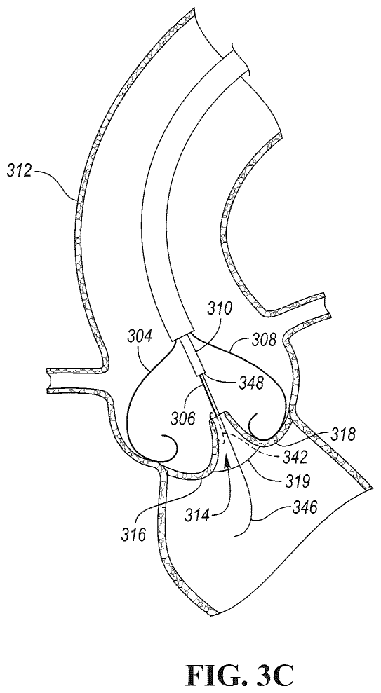

FIG. 3A depicts a cross-sectional view of patient anatomy 300 and a schematic, side view of positioning catheter 302 and positioning arms (i.e., first positioning arm 304, second positioning arm 306, and third positioning arm 308) in a deployed position. FIG. 3B depicts a cross-sectional view of positioning catheter 302 with the positioning arms taken along line 3B-3B of FIG. 3A. FIG. 3C depicts a schematic, side view of guide catheter 310 extending through positioning catheter 302 in the aligned position. In one or more embodiments, positioning catheter 302 is positioned within ascending aorta 312 to align with opening 314 formed by first aortic leaflet 316, second aortic leaflet 318, and third aortic leaflet 319.

As shown in FIG. 3B, first lumen 320, second lumen 322, and third lumen 324 are formed in and are integral to positioning catheter 302. Alternatively, three separate catheters (having three separate lumens) may be secured to the inner wall of positioning catheter 302. As another alternative, the positioning arms may be free floating within the lumen of positioning catheter 302. First positioning arm 304, second positioning arm 306, and third positioning arm 308 extend within first lumen 320, second lumen 322, and third lumen 324, respectively. The location of the positioning arms around the inner surface of positioning catheter 302 is configured to limit obstruction of advancement of guide catheter 310 through positioning catheter 302 by the positioning arms.

As shown in FIG. 3B, the lumen/positioning arm pairs are located an equal radial angle of 120 degrees from each other, although the lumen/positioning arm pairs may be different radial angles from each other in other embodiments. FIG. 3B shows a radial positioning of the lumen/positioning arm pairs at 12 o'clock, 4 o'clock, and 8'clock. While FIG. 3B depicts 3 lumen/positioning arm pairs, in other embodiments, the number of lumen/positioning arm pairs may be 2, 4, 5, or 6, or any range between two of these numbers. Using additional positioning arms may aid in the positioning and aligning functions disclosed herein.

As shown in FIG. 3A, positioning catheter 302 has advanced through the patient's anatomy and is positioned such that distal end 324 of positioning catheter 302 is spaced apart from aortic valve crossing 326. As positioning catheter 302 travels to the position shown in FIG. 3A, the positioning arms are retracted within positioning catheter 302. After positioning catheter 302 reaches the position shown in FIG. 3A, each of the positioning arms may be independently advanced beyond distal end 324 into a deployment position. In another embodiment, the positioning arms may be advanced in unison. The positioning arms may be extended/retracted using an actuator. The actuator may be a slider connected to or part of the proximal ends of the positioning arms. The slider may be translated in a distal direction to actuate the extension of the positioning arms relative to positioning catheter 302. The actuator may be disposed in a handle (not shown) secured to the proximal end (not shown) of positioning catheter 302. In another embodiment, the positioning catheter 302 may be retractable relative to the positioning arms such that retraction of the positioning catheter exposes the positioning arms and the arms expand (e.g., self-expand) to their extended shape. The positioning catheter and the positioning arms may then be advanced in unison to the position in FIG. 3A.

As shown in FIG. 3A, first distal end 328, second distal end 330, and third distal end 332 of the positioning arms extend beyond distal end 324 of positioning catheter 302. First distal end 328, second distal end 330, and third distal end 332 include first outwardly extending portion 334, second outwardly extending portion 336, and third outwardly extending portion 338, respectively, and first inwardly curling portion 340, second inwardly curling portion 342, and third inwardly curling portion 344, respectively. The inwardly curling portions of first distal end 328, second distal end 330, and third distal end 332 are configured to land in the cusp of first aortic leaflet 316, second aortic leaflet 318, and third aortic leaflet 319, respectively, without traumatizing the tissue of the aortic leaflets. The landing of the inwardly curled portions and the extension of the outwardly extending portions collectively form a tripod structure configured to align positioning catheter 302 with opening 314 of aortic valve crossing 326. While the curling portions are inwardly curling relative to a longitudinal axis of positioning catheter 302, in other embodiments, the curled portions may be outwardly curling or a combination of both. As shown in FIG. 3A, each of the curling portions form a single curl, but in other embodiments, multiple curls in two or three dimensions may formed to aid in the atraumatic characteristic of the curling portions.

First distal end 328, second distal end 330, and third distal end 332 may be formed of a flexible material. The flexible material is configured to aid in the landing of the distal portions on the cusps of the aortic leaflets as the aortic leaflets pump blood. The flexible material may be a shape setting metal material configured to form a pre-set shape of the inwardly curling portions and outwardly extending portions. The shape setting material may be Nitinol or a shape setting stainless steel. The inwardly curling portions and outwardly extending portions may be pre-set and then straighten while extending through first lumen 320, second lumen 322, and third lumen 324, and then subsequently curl and outwardly extend, respectively, as they extend beyond distal end 324 of positioning catheter 302. In one or more embodiments, the inwardly curling portions may be coated in a plastic material (e.g., a soft plastic material) to aid in the atraumatic characteristic of the curling portions.

As shown in FIG. 3C, guide catheter 310 extends beyond distal end 325 of positioning catheter 302 after the distal ends of the positioning arms are positioned on the cusps of the aortic leaflets to align guide catheter 314 with opening 314 of aortic valve crossing 326. Guide catheter 314 carries guidewire 346, which is configured to extend beyond distal end 348 of guide catheter 314 and through opening 314 in aortic valve crossing 326. In other embodiments, guidewire 346 may extend into position without guide catheter 314 (e.g., extending directly/only within positioning catheter 302). After guidewire 346 is extended, the positioning arms may be retracted and then positioning catheter 302 may be removed from the vasculature of the patient.

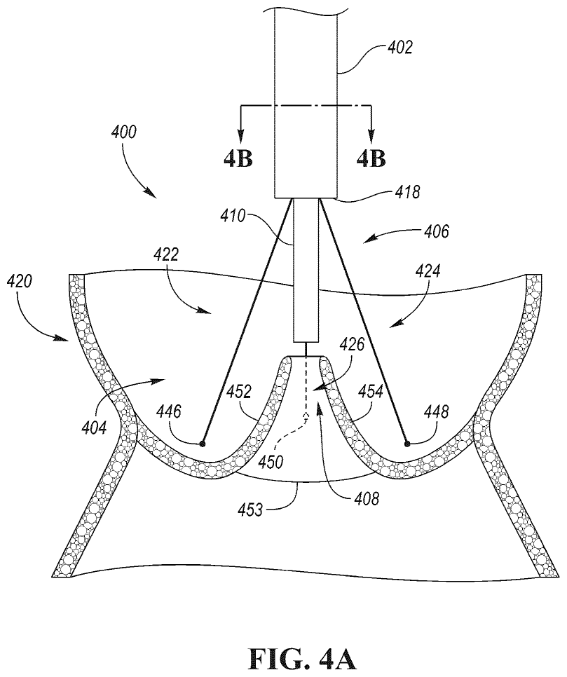

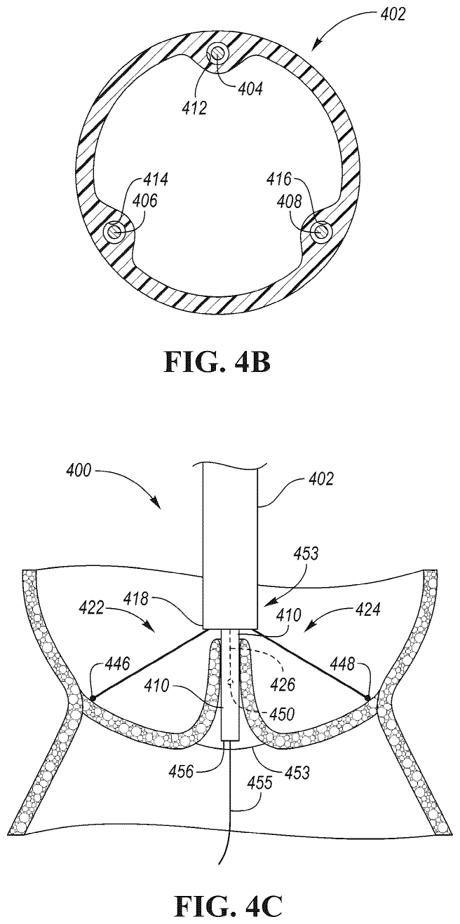

FIG. 4A depicts a cross-sectional view of patient anatomy 400 and a schematic, side view of positioning catheter 402 and positioning arms (i.e., first positioning arm 404, second positioning arm 406, and third positioning arm 408) in a first deployed position. FIG. 4B depicts a cross-sectional view of positioning catheter 402 with positioning arms taken along line 4B-4B of FIG. 4A. FIG. 4C depicts a cross-sectional view of patient anatomy 400 and a schematic, side view of guide catheter 410 extending through positioning catheter 402 in a second deployed position.

As shown in FIG. 4B, first lumen 412, second lumen 414, and third lumen 416 are formed in and are integral to positioning catheter 402. Alternatively, three separate catheters (having three separate lumens) may be secured to the inner wall of positioning catheter 402. As another alternative, the positioning arms may be free floating within the lumen of positioning catheter 402. First positioning arm 404, second positioning arm 406, and third positioning arm 408 extend within first lumen 412, second lumen 414, and third lumen 416, respectively. The location of the positioning arms around the inner surface of positioning catheter 402 is configured to limit obstruction of advancement of guide catheter 410 through positioning catheter 402 by the positioning arms.

As shown in FIG. 4B, the lumen/positioning arm pairs are located an equal radial angle of 120 degrees from each other, although the lumen/positioning arm pairs may be different radial angles from each other in other embodiments. FIG. 4B shows a radial positioning of the lumen/positioning arm pairs at 12 o'clock, 4 o'clock, and 8'clock. While FIG. 4B depicts 3 lumen/positioning arm pairs, in other embodiments, the number of lumen/positioning arm pairs may be 2, 4, 5, or 6, or any range between two of these numbers. Using additional positioning arms may aid in the positioning and aligning functions disclosed herein.

As shown in FIG. 4A, positioning catheter 402 has advanced through the patient's anatomy and is positioned such that distal end 418 of positioning catheter 402 is spaced apart from aortic valve crossing 420. As positioning catheter 402 travels to the position shown in FIG. 4A, the positioning arms are retracted within positioning catheter 402. After positioning catheter 402 reaches the position shown in FIG. 4A, each of the positioning arms may be independently advanced beyond distal end 418 into the first deployed position. In another embodiment, the positioning arms may be advanced in unison. The positioning arms may be extended into the first and second deployed positions using an actuator. The actuator may be a slider connected to or part of the proximal ends of the positioning arms. The slider may be translated in a distal direction to actuate the extension of the positioning arms relative to positioning catheter 402. The actuator may be disposed in a handle (not shown) secured to the proximal end (not shown) of positioning catheter 402. In another embodiment, the positioning catheter 402 may be retractable relative to the positioning arms such that retraction of the positioning catheter exposes the positioning arms and the arms expand (e.g., self-expand) to their extended shape. The positioning catheter and the positioning arms may then be advanced in unison to the position in FIG. 4A.

As shown in FIG. 4A, first distal end 422, second distal end 424, and third distal end 426 of the positioning arms extend beyond distal end 418 of positioning catheter 402. First distal end 422, second distal end 424, and third distal end 426 terminate in first atraumatic end 446, second atraumatic end 448, and third atraumatic end 450, respectively. First atraumatic end 446, second atraumatic end 448, and third atraumatic end 450 are configured to land on cusps of first aortic leaflet 452, second aortic leaflet 454, and third aortic leaflet 453 without traumatizing the tissue of the aortic leaflets.

As shown in FIG. 4C, after the positioning arms reach the first deployed position, positioning catheter 402 may be advanced distally to slide first distal end 422, second distal end 424, and third distal end 426 outwards to spread them further apart. As the distal ends become further apart, the distance between the leaflets decrease, thereby helping to guide a guidewire through the opening in the leaflets. The landing of the atraumatic ends and the distal ends collectively form a tripod configuration to align positioning catheter 402 with opening 453 of aortic valve crossing 420. As shown in FIG. 4C, guide catheter 410 extends beyond distal end 418 of positioning catheter 402 after the atraumatic ends are positioned on the cusps of the aortic leaflets to align guide catheter 410 with opening 453 of aortic valve crossing 420. Guide catheter 410 carries guidewire 455, which is configured to extend beyond distal end 456 of guide catheter 410 and through opening 453 of aortic valve crossing 420. In other embodiments, guidewire 455 may extend into position without guide catheter 410 (e.g., extending directly/only within positioning catheter 402). After guidewire 455 is extended, the positioning arms may be retracted and then positioning catheter 402 may be removed from the vasculature of the patient.

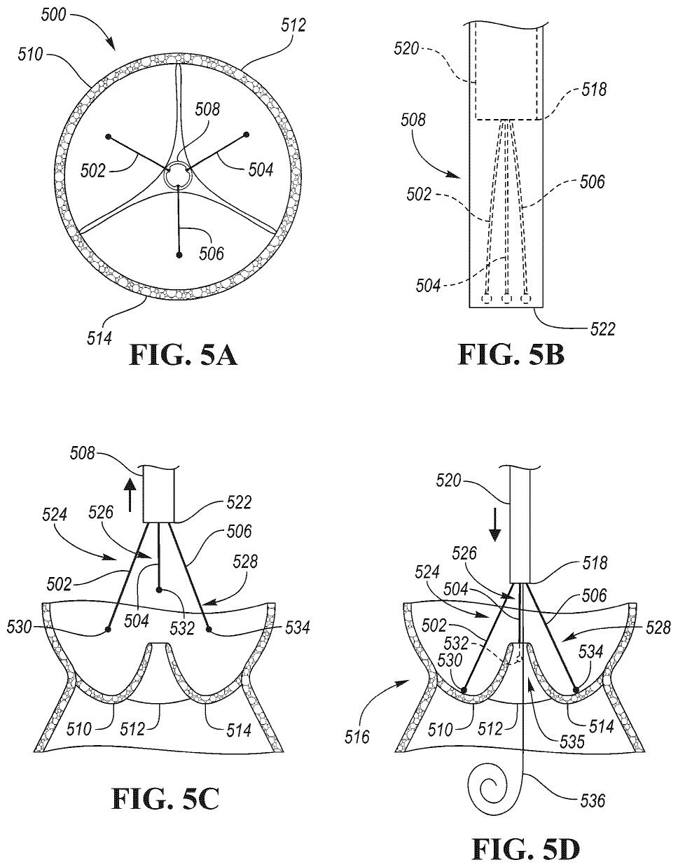

FIG. 5A depicts a top view of aortic valve 500 and positioning arms (i.e., first positioning arm 502, second positioning arm 504, and third positioning arm 506) of positioning sheath 508. Aortic valve 500 includes first aortic leaflet 510, second aortic leaflet 512, and third aortic leaflet 514. FIG. 5B depicts a side view of positioning sheath 508 showing the positioning arms in a constrained position. FIG. 5C depicts a cross-sectional view of aortic valve crossing 516 and a schematic, side view of positioning sheath 508 in a retracted position. FIG. 5D depicts a cross-sectional view of aortic valve crossing 516 and a schematic, side view of the positioning arms extending from distal end 518 of positioning catheter 520 in a deployed position.

As shown in FIG. 5B, the proximal ends of first positioning arm 502, second positioning arm 504, and third positioning arm 506 extend from distal end 518 of positioning catheter 520. Although as shown the positioning arms extend from a single anchor point, the anchor points may instead be around the periphery of distal end 518 of positioning catheter 520. While FIG. 5B depicts 3 positioning arms, in other embodiments, the number of positioning arms may be 2, 4, 5, or 6, or any range between two of these numbers. Using additional positioning arms may aid in the positioning and aligning functions disclosed herein.

In one or more embodiments, positioning sheath 508 advances through the patient's anatomy and is positioned such that distal end 522 of positioning sheath 508 is spaced apart from aortic valve crossing 516. As positioning sheath 508 travels to this position, the positioning arms are retracted within positioning sheath 508. After positioning sheath 508 reaches this position, positioning sheath 508 may be retracted in a proximal axial direction while maintaining the positioning of the positioning arms to expand first distal end 524, second distal end 526, and third distal end 528 of the positioning arms as shown in FIG. 5C. Positioning sheath 508 may be retracted using an actuator. The actuator may be a slider connected to or part of the proximal ends of the positioning arms. The slider may be translated in a distal direction to actuate the extension of the positioning arms relative to positioning sheath 508. The actuator may be disposed in a handle (not shown) secured to the proximal end (not shown) of positioning sheath 508.

First distal end 524, second distal end 526, and third distal end 528 terminate in first atraumatic end 530, second atraumatic end 532, and third atraumatic end 534, respectively. First atraumatic end 530, second atraumatic end 532, and third atraumatic end 534 are configured to land on cusps of first aortic leaflet 510, second aortic leaflet 512, and third aortic leaflet 514, respectively, without traumatizing the tissue of the aortic leaflets.

First distal end 524, second distal end 526, and third distal end 528 may be formed of a flexible material. The flexible material is configured to aid in the landing of the distal portions on the aortic leaflets as the aortic leaflets pump blood. The flexible material may be a shape setting metal material configured to form a pre-set shape of the outwardly extending portions. The shape setting material may be Nitinol or a shape setting stainless steel.

As shown in FIG. 5D, positioning catheter 520 is advanced in a distal axial direction such that first atraumatic end 530, second atraumatic end 532, and third atraumatic end 534 land on first aortic leaflet 510, second aortic leaflet 512, and third aortic leaflet 514, respectively, to align positioning catheter 520 with opening 535 of aortic valve crossing 516. In one or more embodiments, the distal ends of the positioning arms are fixed to distal end 518 of positioning catheter 520. First distal end 524, second distal end 526, and third distal end 528 are in a tripod configuration to form a space within the tripod configuration to accommodate the advancement of guidewire 536 (i.e., a pig tail guidewire as shown in this embodiment). As shown in FIG. 5D, guidewire 536 extends beyond distal end 518 of positioning catheter 520 and through opening 535 of aortic valve crossing 516. After guidewire 536 is extended, positioning sheath 508 may be advanced in a distal axial direction to recapture the positioning arms. After the positioning arms are recaptured, positioning sheath 508 and/or positioning catheter 520 may be removed from the patient's vasculature.

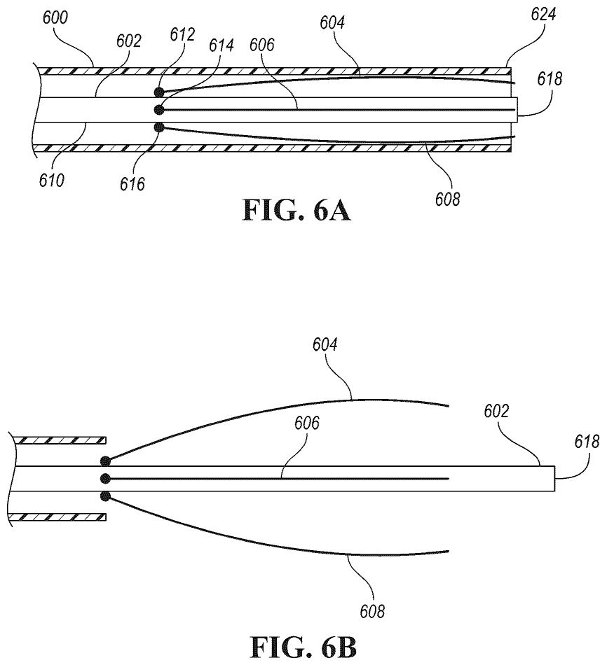

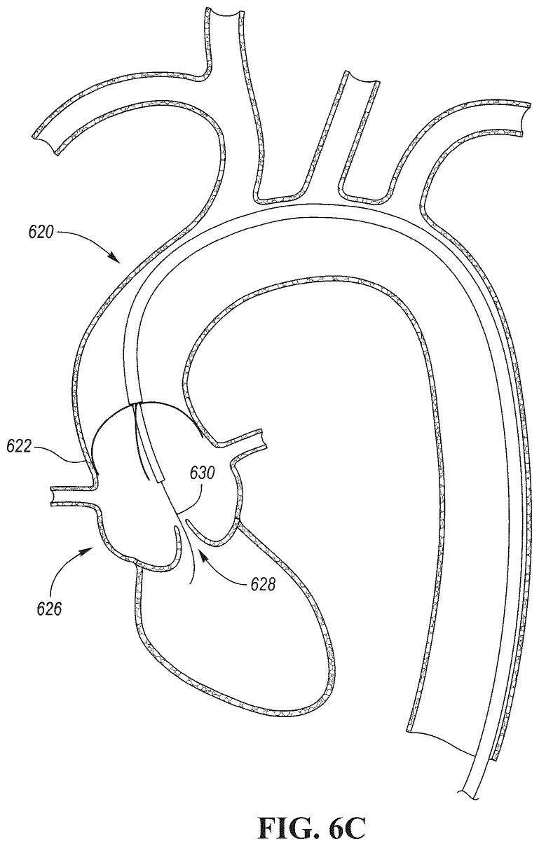

FIG. 6A depicts a cross-sectional view of positioning sheath 600 and a schematic, side view of positioning catheter 602 with first positioning arm 604, second positioning arm 606, and third positioning arm 608 in a constrained position within positioning sheath 600. Positioning catheter 602 may be formed of a braided shaft or a laser cut hypotube. Positioning catheter 602 extends within positioning sheath 600. First positioning arm 604, second positioning arm 606, and third positioning arm 608 are anchored to outer surface 610 of positioning catheter 602 with first anchor 612, second anchor 614, and third anchor 616, respectively. The anchors may mechanically and/or chemically bond the positioning arms to outer surface 610 of positioning catheter 602. A non-limiting example of an anchor is a weld point. The anchors may all be located on a circular path around outer surface 610. The positioning arms in the constrained position may terminate at or near distal end 618 of positioning sheath 600.

FIG. 6B depicts a cross-sectional view of positioning sheath 600 and a schematic, side view of positioning catheter 602 with the positioning arms in a deployed position outside of positioning sheath 600. FIG. 6C depicts a cross-sectional view of patient anatomy 620 and a schematic, side view of positioning catheter 602 with the positioning arms in the deployed position against ascending aorta 622. As shown in FIGS. 6B and 6C, the positioning arms spring outward from outer surface 610 of positioning catheter 602 to form curved portions to atraumatically interact with ascending aorta 622. In this deployed position, the positioning arms terminate short of distal end 618 of positioning catheter 602.

The positioning arms may be formed of a flexible material. The flexible material is configured to aid in the landing of the positioning arms on the vessel wall of ascending aorta 622. The flexible material may be a shape setting metal material configured to form a pre-set shape of the positioning arms in the deployed position. The shape setting material may be Nitinol or a shape setting stainless steel.

The positioning arms may be located an equal radial angle of 120 degrees from each other, although the positioning arm pairs may be different radial angles from each other in other embodiments. The radial positioning may be at 12 o'clock, 4 o'clock, and 8'clock. While FIGS. 6A through 6C show 3 positioning arms, the number of positioning arms may be 2, 4, 5, or 6, or any range between two of those numbers. Using additional positioning arms may aid in the positioning and aligning functions disclosed herein.

In one or more embodiments, positioning sheath 600 advances through the patient's anatomy and is positioned such that distal end 624 of positioning sheath 600 is spaced apart from aortic valve crossing 626. As positioning sheath 600 travels to this position, the positioning arms are retracted within positioning sheath 600. After positioning sheath 600 reaches this position, positioning sheath 600 may be retracted and/or positioning catheter 602 may be advanced to transition the positioning arms to the deployed position. Positioning sheath 600 and/or positioning catheter 602 may be retracted and/or advanced, respectively, using an actuator. The actuator may be disposed in a handle (not shown) secured to the proximal ends (not shown) of positioning sheath 600 and positioning catheter 602.

As shown in FIG. 6C, the positioning arms in the deployed position to align positioning catheter 602 with opening 628 of aortic valve crossing 626. Positioning catheter 602 carries guidewire 630, which is configured to extend beyond distal end 618 of positioning catheter 602 and through opening 628 of aortic valve crossing 626. After guidewire 454 is extended, the positioning arms may be retracted and then positioning sheath 600 and/or positioning catheter 602 may be removed from the vasculature of the patient.

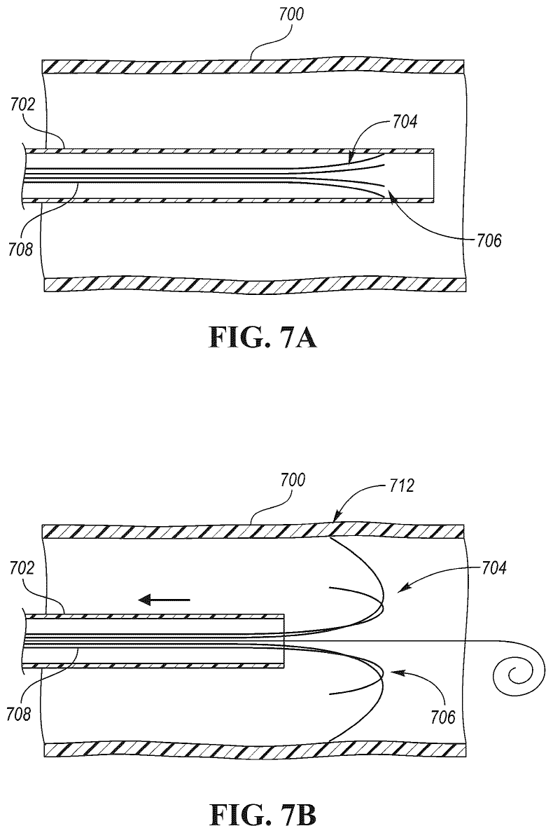

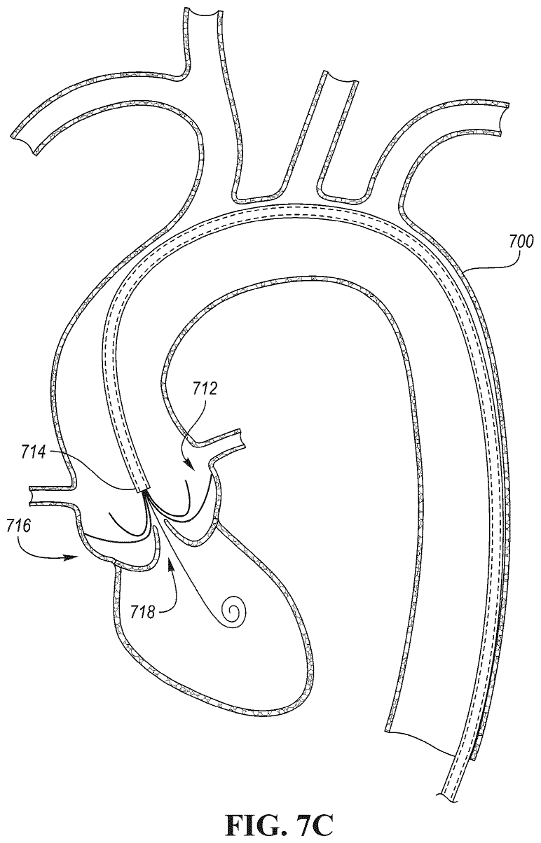

FIG. 7A depicts a cross-sectional view of aortic wall 700 and a schematic, side view of positioning catheter 702 with positioning spindles 704 in a constrained position. Distal ends 706 of positioning spindles 704 may be free from each other while proximal portions 708 of positioning spindles 704 may be intertwined or otherwise meshed or connected. FIG. 7B depicts a cross-sectional view of aortic wall 700 and a schematic, side view of positioning catheter 702 with positioning spindles 704 in a deployed position in which spindles contact a portion of ascending aorta 712. FIG. 7C depicts a cross-sectional view of aortic wall 700 and a schematic, side view of positioning catheter 702 with positioning spindles 704 in the deployed position.

Positioning spindles 704 may be formed of a flexible material. The flexible material is configured to aid in the landing of the positioning arms on the vessel wall of ascending aorta 712. The flexible material may be a shape setting metal material configured to form a pre-set shape of the positioning spindles in the deployed position. The shape setting material may be Nitinol or a shape setting stainless steel. While FIGS. 7A through 7C show 4 positioning spindles, the number of positioning spindles may be 2, 3, 4, 5, 6, 7, or 8, or any range between two of those numbers. Using additional positioning spindles may aid in the positioning and aligning functions disclosed herein. Positioning spindles 704 may terminate in atraumatic ends to atraumatically interact with ascending aorta 712.

In one or more embodiments, positioning catheter 702 advances through the patient's anatomy and is positioned such that distal end 714 of positioning catheter 702 is spaced apart from aortic valve crossing 716. As positioning catheter 702 travels to this position, positioning spindles 704 are retracted within positioning catheter 702. After positioning catheter 702 reaches this position, positioning catheter 702 may be retracted and/or positioning spindles 704 may be advanced to transition positioning spindles 704 to the deployed position. Positioning catheter 702 and/or positioning spindles 704 may be retracted and/or advanced, respectively, using an actuator. The actuator may be disposed in a handle (not shown) secured to the proximal ends (not shown) of positioning catheter 702 and positioning spindles 704.

As shown in FIG. 7C, positioning spindles 704 in the deployed position expand outward and press against aortic wall 700 to align positioning catheter 702 with opening 718 of aortic valve crossing 716. All or less than all of positioning spindles 704 contact aortic wall 700. Positioning catheter 702 carries a guidewire, which is configured to extend beyond distal end 714 of positioning catheter 702 and through opening 718 of aortic valve crossing 716. After the guidewire is extended, positioning spindles may be retracted and then positioning catheter 702 and positioning spindles 704 may be removed from the vasculature of the patient.

While exemplary embodiments are described above, it is not intended that these embodiments describe all possible forms. The words used in the specification are words of description rather than limitation, and it is understood that various changes can be made without departing from the spirit and scope of the disclosure. As previously described, the features of various embodiments can be combined to form further embodiments of the invention that may not be explicitly described or illustrated. While various embodiments could have been described as providing advantages or being preferred over other embodiments or prior art implementations with respect to one or more desired characteristics, those of ordinary skill in the art recognize that one or more features or characteristics can be compromised to achieve desired overall system attributes, which depend on the specific application and implementation. These attributes can include, but are not limited to cost, strength, durability, life cycle cost, marketability, appearance, packaging, size, serviceability, weight, manufacturability, ease of assembly, etc. As such, to the extent any embodiments are described as less desirable than other embodiments or prior art implementations with respect to one or more characteristics, these embodiments are not outside the scope of the disclosure and can be desirable for particular applications.

What is claimed is:

1. An alignment method for aortic valve crossing, the method comprising:
advancing a positioning catheter through an aorta to a delivery position in which a distal end of the positioning catheter is spaced apart from an aortic valve crossing, the positioning catheter defines a positioning lumen containing positioning arms;
landing the positioning arms on cusps of aortic leaflets of the aortic valve crossing to align the positioning catheter with an opening defined by the aortic leaflets; and
extending a longitudinally extending guidewire through the opening.

2. The alignment method of claim 1, wherein the landing step includes extending the positioning arms through the distal end of the positioning catheter such that the positioning arms extend outward the positioning catheter to contact the cusps of the aortic leaflets.

3. The alignment method of claim 2, wherein the extending step including curling the distal ends of the positioning arms to contact the cusps of the aortic leaflets atraumatically.

4. The alignment method of claim 1, wherein the landing step includes advancing the positioning catheter to invert the positioning arms about joints positioned medially the positioning arms.

5. The alignment method of claim 4, wherein the advancement of the positioning catheter extends the longitudinally extending guide through the opening.

6. The alignment method of claim 1, wherein the landing step includes manipulating each of the positioning arms independently to align the positioning catheter with the opening defined by the aortic leaflets.

7. The alignment method of claim 6, wherein the manipulating step includes tensioning the positioning catheter.

8. An alignment method for aortic valve crossing, the method comprising:
advancing a positioning sheath through an aorta to a delivery position in which a distal end of the positioning sheath is spaced apart from an aortic valve crossing, the positioning sheath defines a positioning lumen containing positioning arms;
retracting the positioning sheath to expose the positioning arms;
landing the positioning arms on cusps of aortic leaflets of the aortic valve crossing to align the positioning sheath with an opening defined by the aortic leaflets; and
extending a longitudinally extending guidewire through the opening.

9. The alignment method of claim 8, wherein the positioning arms include proximal ends and distal ends, and the distal ends are attached to a positioning catheter extending within the positioning lumen.

10. The alignment method of claim 9, wherein the landing step includes advancing the positioning catheter such that the positioning arms contact the cusps of the aortic leaflets.

11. The alignment method of claim 8, wherein the longitudinally extending guide wire extends within a guide catheter.

12. The alignment method of claim 8, wherein the positioning arms include a first positioning arm, a second positioning arm, and a third positioning arm.

13. The alignment method of claim 12, wherein the aortic leaflets include a first aortic leaflet, a second aortic leaflet, and a third aortic leaflet, and the landing step includes landing the first positioning arm on a first cusp of the first aortic leaflet, landing the second positioning arm on a second cusp of the second aortic leaflet, and landing the third positioning arm on a third cusp of the third aortic leaflet.

14. The alignment method of claim 12, wherein the landing step includes positioning the first, second, and third positioning arms in a tripod configuration.

15. The alignment method of claim 14, wherein the tripod configuration forms a space for extension of the longitudinally extending guide.

16. An alignment method for aortic valve crossing, the method comprising:
advancing a positioning sheath through an aorta to a delivery position in which a distal end of the positioning sheath is spaced apart from an aortic valve crossing, the positioning sheath defines a positioning lumen containing positioning arms;
landing the positioning arms on a portion of an ascending aorta to align the positioning catheter with an opening defined by aortic leaflets of the aortic valve crossing; and
extending a longitudinally extending guide through the opening.

17. The alignment method of claim 16, wherein the positioning arms are anchored to an outer surface of a positioning catheter, and the landing step includes differentially moving the positioning sheath or the positioning catheter such that the positioning arms extend outward the positioning catheter to contact the portion of the ascending aorta.

18. The alignment method of claim 17, wherein the differentially moving step includes advancing the positioning catheter relative to the positioning sheath.

19. The alignment method of claim 17, wherein the differentially moving step includes retracting the positioning sheath relative to the positioning catheter.

20. The alignment method of claim 16, wherein the positioning arms include distal ends having spindles.
